(19) United States
(12) Patent Application Publication
(10) Pub. No.: US 2024/0239966 A1
(43) Pub. Date: Jul. 18, 2024

(54) COMPOSITION FOR PREPARING MULTI-CROSSLINKED TEMPERATURE-SENSITIVE HYDROGEL, AND USE THEREOF

(71) Applicant: MEDIFAB CO., LTD., Seoul (KR)

(72) Inventors: Mi Sun CHA, Seoul (KR); Su Hee KIM, Seoul (KR); Soo Hee LEE, Seoul (KR)

(21) Appl. No.: 18/577,650

(22) PCT Filed: Jul. 7, 2022
(86) PCT No.: PCT/KR2022/009842

§ 371 Date: Apr. 1, 2024

(30) Foreign Application Priority Data
Jul. 7, 2021 (KR) 10-2021-0089206

(51) Int. Cl. C08J 3/075; A61L 27/20; A61L 27/36; A61L 27/52; C08K 3/32; C08K 5/053

(52) U.S. Cl. CPC C08J 3/075; A61L 27/20; A61L 27/3683; A61L 27/52; C08K 3/32; C08K 5/053; A61L 2400/06; C08J 2305/08

(57) ABSTRACT

The present application relates to a composition for preparing a temperature-sensitive hydrogel and a use thereof, and provides a temperature-sensitive hydrogel composition and a method for preparing a temperature-sensitive hydrogel composition using the composition, including a first agent in a liquid formulation including an aqueous solution containing chitosan and phosphate ions, including an aqueous solution containing 0.05 wt % to 3.5 wt % of chitosan and 0.1 wt % to 40 wt % of phosphate ions, based on the total weight of the first agent composition.

### TECHNICAL FIELD

The present application claims priority to Korean Patent Application No. 10-2021-0089206, filed on Jul. 7, 2021, which is hereby incorporated by reference in its entirety. The present disclosure relates to a composition for preparing a multi-crosslinked temperature-sensitive hydrogel and use thereof.

### BACKGROUND ART

A tissue of a human skin maintain the structure by an extracellular matrix including protein such as a collagen, elastin and glycosaminoglycan. A Skin tissue defect may occur due to external shock, disease, surgery, or aging. Therefore, skin tissue augmentation with a biomaterial has been used for medical and cosmetic purposes. Such augmentation has been done surgically through plastic surgery, or by injecting or attaching biological tissue or synthetic polymer chemical substance to a relevant area to recover, restore, or correct skin tissue.

Among the biomaterial used for skin tissue augmentation, a substance with a component similar to skin tissue and is inserted into a specific area to expand soft tissue to augment the volume of cheeks, lips, breasts, buttocks, etc. for cosmetic purposes, and is used for wrinkle improvement or contour correction by reducing fine lines and deep wrinkles in the skin is called a soft tissue augmentation material, generally referred to as a dermal filler.

Adhesion is a phenomenon in which a surrounding organ or tissue stick together with a wounded area due to excessive generation of fibrous tissue or coagulation of blood in a healing process of a wound formed inside the skin or abdominal cavity after a surgical operation. In the past, an anti-adhesive agent has been used as the biomaterial to prevent adhesion from occurring between a surgical site and normal tissue by forming a physical barrier in the area where adhesion is expected.

In addition, among the biomaterial, a wound dressing may promote recovery and healing by providing a moist environment to the damaged area when skin tissue is damaged due to external shock, disease, surgery, or aging.

Among a polymer that may be used in such biomaterial, a hydrogel refers to a substance with a polymer network structure containing a large amount of moisture and is formed by a homopolymer or a copolymer, etc. The hydrogel made from the synthetic polymer has strong bonding forces, such as a chemical covalent bond, between molecules through cross-linking, and has a strong mechanical property unlike a natural polymer, so it is relatively rare for the hydrogel shape to be damaged by external stimuli such as temperature and external force, etc. However, in the case of the hydrogel based on the synthetic polymer, improvements are needed in terms of biocompatibility, efficacy maintenance, and differences, etc. in manufacturing methods depending on an indication.

These synthetic polymer-based hydrogels have different physical properties such as filler viscosity, elasticity, and stability in the body depending on a cross-linking rate. Therefore, research and development on technologies that may be used according to indications and other uses by changing the physical properties of the biomaterial is being actively conducted (Korean Patent Registration 10-2100506), but is still insufficient. For example, in the case of an indication that require a gel with a high physical property, there is a problem that the application is limited because an injection force is too high. Therefore, there is a need to develop a new concept of a next-generation biomaterial that maximizes the advantages of formulations using existing natural/synthetic polymers while complementing the shortcomings, has high stability in the body, and may change viscosity and elasticity to an appropriate level depending on the indication.

### DISCLOSURE

### Technical Problem

One aspect provides a composition for preparing a temperature-sensitive hydrogel composition including a first agent in a liquid formulation including chitosan and an aqueous solution containing phosphate ions, wherein, based on the total weight of the first agent composition, the composition includes from 0.05 to 3.5 wt % chitosan and from 0.1 to 40 wt % an aqueous solution containing phosphate ions.

Another aspect provides a method of preparing the temperature-sensitive hydrogel composition, including a phase of mixing a solution including chitosan ions and the aqueous solution containing phosphate ions to prepare the first agent in the liquid formulation, and stabilizing the first agent in the liquid formulation.

Another aspect provides the temperature-sensitive hydrogel composition prepared by the above method.

Another aspect provides a method of treating a patient using the temperature-sensitive hydrogel prepared by the method.

### Technical Solution

One aspect is to provide a composition for preparing a composition for preparing a temperature-sensitive hydrogel composition including a first agent in a liquid formulation including an aqueous solution containing chitosan and phosphate ions.

As used herein, the term “hydrogel” may refer to a three-dimensional reticular structure made of hydrophilic polymers crosslinked by covalent or non-covalent bonds. Due to a hydrophilic nature of a component substance, the hydrogel may absorb large amounts of water in an aqueous solution and in an aqueous environment and swell, but has a property of not dissolving due to its crosslinked structure. Therefore, hydrogels with various shapes and properties may be created depending on the component and manufacturing method, and because the hydrogel generally contain a large amount of moisture, the hydrogel may have properties intermediate between liquid and solid.

As an example, the term “hydrogel” may be used interchangeably with the terms “biomaterial for tissue repair” or “biomaterial composition for tissue repair”.

In an example, the hydrogel may be a tissue repair biomaterial, which is a substance used for tissue repair. For example, the hydrogel may refer to a soft tissue-like filling substance injected into wrinkled skin or an area in need of volume, an anti-adhesion substance between a surgical site and normal tissue, a tissue adhesion substance, a wound dressing substance for artificial skin, etc. The hydrogel may be applied to areas of a body, for example, glabella, forehead, eye bags, crow's feet, nasolabial folds, cheeks, corners of a mouth, chin, etc. The hydrogel is a substance that is directly applied to a human body and must be biocompatible. For example, if the hydrogel is filled in an area where volume is required, the hydrogel needs to have excellent retention/persistence of the gel shape so that the hydrogel may form volume for a long period of time after injection. If the hydrogel is being used to prevent adhesions at a surgical site, the hydrogel must have tissue compatibility at a wound site and low or no cytotoxicity. If used as a wound dressing substance, the hydrogel needs to have excellent retention/persistence to remain attached and applied. In addition, depending on a condition of the hydrogel procedure, the skin surface may become uneven or the procedure result may be undesirable, so the hydrogel formed in the body must be easily degraded or transformed.

As used herein, the term “tissue repair” refers to restoring a structure and function of damaged or aged tissue, and may include, for example, but is not limited to, use as a cosmetic filler, use as an anti-adhesive agent, use as an adhesive, use as a wound dressing, use as a cosmetic implant, etc.

As used herein, the term “temperature-sensitive” refers to a physical property that causes the formulation to change with room temperature, and may refer to a property that exists in a liquid formulation, in other words, a sol, at room temperature conditions, for example, 4 to 25° C., but converts to a gel form, for example, at 25 to 60° C.

As used herein, the term “multi-crosslink” refers to crosslinking multiple times using one or more crosslinking agents (for example, phosphate ions, glycerol, or a combination thereof), such that a mechanical property and degradation rate, etc., of the hydrogel may be controlled by a multi-crosslink using one or more crosslinking agents. Viscoelasticity and degradation rate may be controlled by variously adjusting a weight ratio of one or more cross-linking agents included in the hydrogel, which may be used to control injection force and for various indications.

In an example, the multi-crosslink may be performed by primary crosslinking the gelated material in vitro and then injecting the material into the body, followed by secondary crosslinking under an in vivo condition. For example, for an indication requiring a hydrogel with a high physical property, the injection force may be controlled through primary crosslinking, and a secondary crosslinking may be performed in vivo to achieve the high physical property.

As used herein, “in vivo condition” is not limited to a condition under which secondary crosslinking may occur, such as a body temperature of a subject, body fluid component, body pH, body salinity, etc. For example, the in vivo condition may be a basic condition.

In an embodiment, the first agent may be a composition in a liquid formulation including an aqueous solution containing a chitosan and phosphate ions.

As used herein, the term “chitosan” may refer to a linear polysaccharide consisting of D-glucosamine and N-acetylglucosamine. The chitosan may be represented by Structural Formula 1 below and may be obtained by, but is not limited to, treating crab, shrimp and crustacean shells with a sodium hydroxide base. The chitosan may include a chitosan derivative in addition to pure chitosan. For example, the chitosan derivative may include at least either one of phthalated chitosan, esterified chitosan, amidated chitosan, or formylated chitosan:

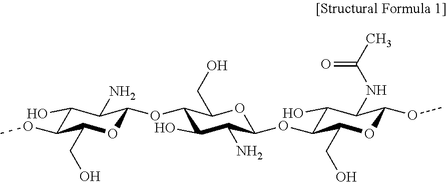

As used herein, the term “phosphate ions” is a component that bind to an amine group of chitosan and contributes to strengthening the strength of the temperature-sensitive hydrogel formed from the first agent and under a room temperature condition, and may be provided, for example, in the form of an aqueous solution containing phosphate ions. The aqueous solution containing phosphate ions may include at least one or more phosphate selected from the group consisting of, for example, sodium phosphate dibasic, sodium phosphate monobasic, ammonium phosphate dibasic, dihydrogen phosphate, trisodium phosphate, potassium phosphate dibasic, potassium phosphate monobasic, dimethyl phosphate, monomagnesium phosphate, magnesium phosphate dibasic, lithium dihydrogen phosphate, lithium phosphate, calcium dihydrogen phosphate hydrate, and calcium hydrogen phosphate, but the aqueous solution containing phosphate ions may be extended and applied without limitation as long as it is a substance that may provide a phosphate group or phosphate that may bind to the amine group of chitosan.

The aqueous solution containing chitosan may be an aqueous solution containing 0.01 to 5 wt %, 0.01 to 4 wt %, 0.05 to 4 wt %, or 0.05 to 3.5 wt % of chitosan, based on the total weight of a first agent composition.

For example, the content of the aqueous solution containing chitosan may be 0.05 to 3.5 wt %, based on the total weight of the first agent composition. In this case, if the content of the aqueous solution containing chitosan is less than the above range, there is a problem that the chitosan content is low and the persistence in the body is poor, and if the content of the aqueous solution containing chitosan is more than the above range, there is a problem that the chitosan is likely to precipitate and the filtering process is difficult due to the high viscosity.

The aqueous solution containing phosphate ions may be an aqueous solution containing phosphate ions in an amount of 0.01 to 50 wt %, 0.01 to 45 wt %, 0.01 to 40 wt %, or 0.1 to 40 wt %, based on the total weight of the first agent composition.

For example, the content of the aqueous solution containing phosphate ions may be from 0.1 to 40 wt %, based on the total weight of the first agent composition. In this case, if the content of the aqueous solution containing phosphate ions is less than the above range, there is a problem that the amount of phosphate ions for cross-linking is insufficient to form a hydrogel, and if the content of the aqueous solution containing phosphate ions is more than the above range, the cross-linking reaction proceeds at room temperature, and the chitosan may precipitate due to the increase in pH while losing the temperature-sensitive feature that allows injection under a room temperature condition.

A composition according to an aspect may include chitosan and phosphate ions, wherein the chitosan is crosslinked by phosphate ions. The chitosan may be such that the chitosan is crosslinked with phosphate ions to form a chitosan polymer. The crosslinking may be a covalent bond or a non-covalent bond. In an embodiment, a non-covalent bond may be formed by the chitosan phosphate ions.

In other words, the temperature-sensitive hydrogel composition according to an aspect may adjust the physical property (viscosity, strength, etc.) of the hydrogel composition by adjusting the type and content of phosphate ions as the chitosan form the covalent bond and/or non-covalent bond with phosphate ions. Therefore, the physical property of the hydrogel composition may be adjusted and used depending on the tissue repair use of the hydrogel composition.

The first agent may be stabilized at a room temperature condition. For example, the first agent may be exposed to the room temperature condition for 10 days or more, specifically, but not limited to, 1 to 10 days, 12 hours to 10 days, 1 day to 10 days, 1 day to 7 days, 10 to 14 days, 10 to 21 days, 10 to 28 days, 10 to 35 days, 10 to 42 days, 15 to 21 days, 15 to 28 days, 15 to 35 days, 15 to 42 days, 20 to 28 days, 20 to 35 days, or 20 to 42 days. The stabilization process may contribute to controlling the physical property of the hydrogel by adjusting the level of ionic bonding between the amine group and phosphate ions of chitosan in the first agent.

In an embodiment, the temperature-sensitive hydrogel composition according to an aspect may be prepared by mixing an aqueous solution containing chitosan containing 1.5 to 3.5% chitosan and the aqueous solution containing phosphate ions. In an example, the first agent, may include 0.01 to 0.3 parts by weight, 0.02 to 0.3 parts by weight, 0.03 to 0.3 parts by weight, 0.04 to 0.3 parts by weight, 0.05 to 0.3 parts by weight, 0.06 to 0.3 parts by weight, 0.07 to 0.3 parts by weight, 0.08 to 0.3 parts by weight, 0.09 to 0.3 parts by weight, 0.1 to 0.3 parts by weight, 0.1 to 0.25 parts by weight, 0.1 to 0.2 parts by weight, 0.1 to 0.19 parts by weight, 0.1 to 0.18 parts by weight, 0.11 to 0.18 parts by weight, 0.115 to 0.175 parts by weight, 0.12 to 0.17 parts by weight, 0.12 to 0.15 parts by weight, 0.12 to 0.14 parts by weight, 0.12 to 0.13 parts by weight, 0.13 to 0.18 parts by weight, 0.13 to 0.17 parts by weight, 0.13 to 0.16 parts by weight, 0.13 to 0.15 parts by weight, 0.14 to 0.19 parts by weight, 0.15 to 0.19 parts by weight, 0.16 to 0.19 parts by weight, or 0.16 to 0.18 parts by weight of an aqueous solution containing phosphate ions per 1 part by weight of the aqueous solution containing chitosan. In an example, the first agent may include 0.126 to 0.169 parts by weight of an aqueous solution containing sodium phosphate dibasic per 1 part by weight of an aqueous solution containing 2.5% chitosan.

In an embodiment, a weight ratio of the aqueous solution containing chitosan and the aqueous solution containing phosphate ions may be 1:0.10 or more but less than 0.135, 1:0.10 or more but less than 0.13, 1:0.101 or more but less than 0.13, 1:0.102 or more but less than 0.13, 1:0.103 or more but less than 0.13, 1:0.104 or more but less than 0.13, 1:0.105 or more but less than 0.13, 1:0.106 or more but less than 0.13, 1:0.107 or more but less than 0.13, 1:0.108 or more but less than 0.13, 1:0.109 or more but less than 0.13, 1:0.11 or more but less than 0.13, 1:0.111 or more but less than 0.13, 1:0.112 or more but less than 0.13, 1:0.113 or more but less than 0.13, 1:0.114 or more but less than 0.13, 1:0.115 or more but less than 0.13, 1:0.116 or more but less than 0.13, 1:0.117 or more but less than 0.13, 1:0.118 or more but less than 0.13, 1:0.119 or more but less than 0.13, 1:0.12 or more but less than 0.13, 1:0.121 or more but less than 0.13, 1:0.122 or more but less than 0.13, 1:0.123 or more but less than 0.13, 1:0.124 or more but less than 0.13, 1:0.11 to 0.13, 1:0.11 to 0.129, 1:0.11 to 0.128, 1:0.11 to 0.127, 1:0.115 to 0.13, 1:0.115 to 0.129, 1:0.115 to 0.128, 1:0.115 to 0.127, 1:0.12 to 0.13, 1:0.12 to 0.129, 1:0.12 to 0.128, 1:0.12 to 0.127, or 1:0.123 to less than 0.129.

In an embodiment, the weight ratio of the aqueous solution containing chitosan and the aqueous solution containing phosphate ions may be 1:0.13 or more but less than 0.165, 1:0.13 or more but less than 0.16, 1:0.13 or more but less than 0.155, 1:0.131 or more but less than 0.15, 1:0.132 or more but less than 0.15, 1:0.133 or more but less than 0.15, 1:0.134 or more but less than 0.15, 1:0.135 or more but less than 0.15, 1:0.13 or more but less than 0.145, 1:0.131 or more but less than 0.145, 1:0.132 or more but less than 0.145, 1:0.133 or more but less than 0.145, 1:0.134 or more but less than 0.145, 1:0.135 or more but less than 0.145, 1:0.13 or more but less than 0.14, 1:0.131 or more but less than 0.14, 1:0.132 or more but less than 0.14, 1:0.133 or more but less than 0.14, 1:0.134 or more but less than 0.14, 1:0.135 or more but less than 0.14, 1:0.13 or more but less than 0.139, 1:0.131 or more but less than 0.139, 1:0.132 or more but less than 0.139, 1:0.133 or more but less than 0.139, 1:0.134 or more but less than 0.139, 1:0.135 or more but less than 0.139, 1:0.13 or more but less than 0.138, 1:0.131 or more but less than 0.138, 1:0.132 or more but less than 0.138, 1:0.133 or more but less than 0.138, 1:0.134 or more but less than 0.138, 1:0.135 or more but less than 0.138, 1:0.13 or more but less than 0.137, 1:0.131 or more but less than 0.137, 1:0.132 or more but less than 0.137, 1:0.133 or more but less than 0.137, 1:0.134 or more but less than 0.137, or 1:0.135 or more but less than 0.137.

In an embodiment, the weight ratio of the aqueous solution containing the chitosan and the aqueous solution containing phosphate ions may be 1:0.14 to 0.19, 1:0.14 to 0.18, 1:0.14 to 0.17, 1:0.145 to 0.19, 1:0.145 to 0.18, 1:0.145 to 0.17, 1:0.15 to 0.19, 1:0.15 to 0.18, 1:0.15 to 0.17, 1:0.155 to 0.19, 1:0.155 to 0.18, 1:0.155 to 0.17, 1:0.16 to 0.19, 1:0.16 to 0.18, 1:0.16 to 0.17, 1:0.161 to 0.17, 1:0.162 to 0.17, 1:0.163 to 0.17, 1:0.164 to 0.17, 1:0.165 to 0.17, 1:0.166 to 0.17, 1:0.167 to 0.17, or 1:0.168 to 0.17.

In an embodiment, when the weight ratio of the aqueous solution containing chitosan and the aqueous solution containing phosphate ions fall in the range of 1:0.12 or more but less than 0.13, a composite viscosity value, that refers to an elasticity level of the hydrogel composition, may be adjusted to a level of 500 Pas or less, whereby a composition prepared as described above may be utilized as a restorative composition of a fine soft tissue, such as a periorbital filler, a wound dressing, and a material for an injectable/applied wound dressing. For example, when the weight ratio of the aqueous solution containing chitosan and the aqueous solution containing phosphate ions fall in the range of 1:0.13 or more but less than 0.15, the composite viscosity value of the hydrogel composition may be adjusted to a level in the range of 500 Pas to 3000 Pa·s, and the composition prepared as described above may be utilized as a restorative composition for soft tissues of the facial area, a synovial fluid substitute, a wound dressing, or an adhesive wound dressing. For example, when the weight ratio of the aqueous solution containing chitosan and the aqueous solution containing phosphate ions fall in the range of 1:0.16 to 0.18, the composite viscosity value of the hydrogel composition may be adjusted to a level in the range of 3000 Pas to 7000 Pas, and the composition prepared as described above may be utilized as a restorative composition for tissues such as the bridge of the nose or forehead, or as a material for an adhesive wound dressing.

In other words, the temperature-sensitive hydrogel composition according to an aspect may be used to adjust the physical property (viscosity, strength, etc.) of the hydrogel composition by adjusting the volume ratio between the solutions including chitosan or phosphate ions, and may be used by adjusting the physical property of the hydrogel composition according to the tissue repair use of the hydrogel composition. Through the composition of each component mentioned above, the physical property may be maintained stably for a longer period of time.

For example, the first agent composition may undergo a stabilization period of exposure to a room temperature condition for 10 days or more, or may undergo a stabilization period of 1 day to 7 days or less at the room temperature condition. The stabilization period allows for stable physical property to be maintained stably over a longer period of time.

The first agent composition may exhibit a pH of 5.0 to 8.0, pH 5.5 to 8.0, pH 5.5 to 7.5, or pH 5.5 to 7.0. If the pH is greater than the above range, the biomaterial may precipitate, and if the pH is less than the above range, the skin pH may be affected.

The first agent composition may further include a decellularized matrix.

As used herein, the term “decellularized matrix” may be used interchangeably with “decellularized tissue”, “decellularized extracellular matrix” or “decellularized material”. The decellularized matrix refers to a removal of other cellular component, for example, nuclei, cell membrane, and nucleic acid, other than the extracellular matrix, by performing decellularization on tissue and organ of human or animal such as a pig or cattle, etc. This decellularized extracellular matrix, the decellularized matrix, may provide a more natural biomimetic microenvironment for cells to grow and differentiate.

As used herein, the term “extracellular matrix (ECM)” refers to a complex assembly of a biopolymer that fills the intratissue or extracellular space of a tissue. The extracellular matrix is composed of various types of molecules synthesized by cells and secreted and accumulated outside the cell, such as a fibrous protein, complex protein such as a proteoglycan, and cell adhesion protein such as fibronectin and laminin. Thus, the extracellular matrix may vary in component depending on the type of cell from which the extracellular matrix is derived or the degree of differentiation of the cell.

In an example, the decellularized matrix may include from 0.05 to 20 wt %, 0.06 to 20 wt %, 0.07 to 19 wt %, 0.08 to 18 wt %, 0.09 to 17 wt %, 0.1 to 16 wt %, 0.15 to 15 wt %, or 0.20 to 15 wt %, based on the total weight of the first agent composition.

The decellularized matrix may be derived from, but not limited to, skin tissue, cardiac tissue, or adipose tissue. In addition, the bio-environment sensitive hydrogel composition may include one or more selected from the group consisting of a skin tissue-derived decellularized matrix, a cardiac tissue-derived decellularized matrix, and an adipose tissue-derived decellularized matrix.

As an example, the decellularized matrix may have the weight ratio of the cardiac tissue-derived decellularized matrix to the adipose tissue-derived decellularized matrix of 1:0.01 to 1, 1:0.1 to 0.9, 1:0.1 to 0.8, 1:0.1 to 0.7, 1:0.1 to 0.6, 1:0.1 to 0.5, 1:0.1 to 0.4, 1:0.15 to 0.4, 1:0.15 to 0.35 or 1:0.2 to 0.3, preferably 1:0.25.

Furthermore, the decellularized matrix may have the weight ratio of the cardiac tissue-derived decellularized matrix to the skin tissue-derived decellularized matrix of 1:0.01 to 1, 1:0.1 to 0.9, 1:0.1 to 0.8, 1:0.1 to 0.8, 1:0.1 to 0.7, 1:0.1 to 0.6, 1:0.1 to 0.5, 1:0.1 to 0.4, 1:0.15 to 0.4, 1:0.15 to 0.35, or 1:0.2 to 0.3, preferably 1:0.25.

When the decellularized matrix is included in a weight ratio within the above range, the decellularized matrix shows excellent ability to induce vascularized adipose tissue, but when the weight ratio is outside the range, the ability to induce vascularized adipose tissue may be significantly reduced.

Meanwhile, the decellularized matrix may be dissolved in an acidic solution including an enzyme to increase dispersion. The acidic solution may have a pH of 3 to 6.5, a pH of 3.5 to 6.5, a pH of 4 to 6.5, a pH of 4.5 to 6.5, a pH of 5 to 6.5, a pH of 5.2 to 6.3, a pH of 5.4 to 6.1, a pH of 5.6 to 5.9, but is not limited thereto.

The enzyme may be a protease that degrades proteins, such as, but not limited to, pepsin, peptidase, trypsin, or papain, preferably pepsin, but not limited to.

In an embodiment, the composition for preparing the multi-crosslinked temperature-sensitive hydrogel composition may further include a second agent in a liquid formulation containing glycerol.

The second agent may be mixed with the stabilized first agent to adjust the level of covalent bond or non-covalent bond in the mixture, thereby granting a viscoelastic property to the hydrogel.

The second agent may contain a glycerol stock solution or an aqueous solution containing glycerol, for example, if the glycerol stock solution is included, the second agent may contain 100% glycerol at 100 wt %.

In an embodiment, the covalent bond may be formed by the chitosan and glycerol. In another embodiment, a non-covalent bond may be formed by the chitosan phosphate ions. Thus, the chitosan polymer may include both the non-covalent bond with a phosphate group and the covalent bonds with glycerol.

In other words, the temperature-sensitive hydrogel composition according to an aspect may adjust the physical property (viscosity, strength, etc.) of the composition by adjusting the type and content of the crosslinking agent as the chitosan form the covalent bond and/or non-covalent bond with the glycerol and/or phosphate ions.

In an example, the second agent may include 0.01 to 0.3 parts by weight, 0.01 to 0.25 parts by weight, 0.01 to 0.2 parts by weight, 0.01 to 0.15 parts by weight, 0.01 to 0.10 parts by weight, 0.01 to 0.08 parts by weight, 0.01 to 0.07 parts by weight, 0.01 to 0.06 parts by weight, 0.01 to 0.05 parts by weight, 0.01 to 0.04 parts by weight, 0.015 to 0.2 parts by weight, 0.015 to 0.15 parts by weight, 0.015 to 0.10 parts by weight, 0.01 to 0.015 to 0.08 parts by weight, 0.015 to 0.07 parts by weight, 0.015 to 0.06 parts by weight, 0.015 to 0.05 parts by weight, 0.015 to 0.04 parts by weight, 0.015 to 0.03 parts by weight, 0.02 to 0.10 parts by weight, 0.02 to 0.08 parts by weight, 0.02 to 0.07 parts by weight, 0.02 to 0.05 parts by weight, 0.02 to 0.04 parts by weight, 0.02 to 0.03 parts by weight, 0.03 to 0.10 parts by weight, 0.03 to 0.09 parts by weight, 0.03 to 0.08 parts by weight, 0.03 to 0.07 parts by weight, 0.03 to 0.065 parts by weight, 0.04 to 0.10 parts by weight, 0.04 to 0.09 parts by weight, 0.04 to 0.08 parts by weight, 0.04 to 0.07 parts by weight, 0.04 to 0.065 parts by weight, 0.05 to 0.10 parts by weight, 0.05 to 0.09 parts by weight, 0.05 to 0.08 parts by weight, 0.05 to 0.08 parts by weight, 0.05 to 0.07 parts by weight, 0.05 to 0.065 parts by weight, 0.06 to 1.0 parts by weight, 0.06 to 0.09 parts by weight, 0.06 to 0.08 parts by weight, 0.06 to 0.07 parts by weight, 0.06 to 0.065 parts by weight, 0.07 to 0.15 parts by weight, 0.07 to 0.14 parts by weight, 0.07 to 0.13 parts by weight, 0.08 to 0.15 parts by weight, 0.08 to 0.14 parts by weight, 0.08 to 0.13 parts by weight, 0.09 to 0.15 parts by weight, 0.09 to 0.14 parts by weight, 0.09 to 0.13 parts by weight, 0.10 to 0.15 parts by weight, 0.10 to 0.14 parts by weight, 0.10 to 0.13 parts by weight, 0.11 to 0.15 parts by weight, 0.11 to 0.14 parts by weight, 0.11 to 0.13 parts by weight, 0.12 to 0.15 parts by weight, 0.12 to 0.14 parts by weight, or 0.12 to 0.13 parts by weight of glycerol per 1 part by weight of the aqueous solution containing chitosan of the first agent. In an example, the second agent may include 0.024 to 0.122 parts by weight of 100% glycerol per 1 part by weight of the aqueous solution containing 2.5% chitosan of the first agent.

In an embodiment, in the composition for preparing the multi-crosslinked temperature-sensitive hydrogel composition, the second agent may include 0.001 to 0.06 parts by weight, 0.001 to 0.05 parts by weight, 0.001 to 0.04 parts by weight, 0.001 to 0.05 parts by weight, 0.001 to 0.04 parts by weight, 0.001 to 0.03 parts by weight, 0.01 to 0.06 parts by weight, 0.01 to 0.05 parts by weight, 0.01 to 0.04 parts by weight, 0.01 to 0.03 parts by weight, 0.015 to 0.06 parts by weight, 0.015 to 0.05 parts by weight, 0.015 to 0.04 parts by weight, 0.015 to 0.03 parts by weight, 0.02 to 0.06 parts by weight, 0.02 to 0.05 parts by weight, 0.02 to 0.04 parts by weight, 0.02 to 0.03 parts by weight, 0.021 to 0.06 parts by weight, 0.021 to 0.05 parts by weight, 0.021 to 0.04 parts by weight, 0.021 to 0.03 parts by weight, 0.022 to 0.06 parts by weight, 0.022 to 0.05 parts by weight, 0.022 to 0.04 parts by weight, 0.022 to 0.03 parts by weight, 0.023 to 0.06 by weight, 0.023 to 0.05 by weight, 0.023 to 0.04 by weight, 0.023 to 0.03 by weight, 0.02 to 0.029 by weight, 0.02 to 0.028 by weight, 0.02 to 0.027 by weight, 0.02 to 0.026 by weight, 0.02 to 0.025 by weight, or 0.022 to 0.026 of glycerol per 1 part by weight of the aqueous solution containing chitosan of the first agent.

In an embodiment, in the composition for preparing the multi-crosslinked temperature-sensitive hydrogel composition, the second agent may include 0.025 parts by weight to 0.120 parts by weight, 0.03 parts by weight to 0.115 parts by weight, 0.03 parts by weight to 0.11 by weight, 0.03 parts by weight to 0.105 by weight, 0.03 parts by weight to 0.1 by weight, 0.03 parts by weight to 0.09 by weight, 0.03 parts by weight to 0.08 by weight, 0.03 parts by weight to 0.07 by weight, 0.04 parts by weight to 0.115 by weight, 0.04 parts by weight to 0.11 by weight, 0.04 parts by weight to 0.105 by weight, 0.04 parts by weight to 0.1 parts by weight, 0.04 parts by weight to 0.09 parts by weight, 0.04 parts by weight to 0.08 parts by weight, 0.04 parts by weight to 0.07 parts by weight, 0.05 parts by weight parts by weight to 0.115 parts by weight, 0.05 parts by weight parts by weight to 0.11 parts by weight, 0.05 parts by weight parts by weight to 0.105 parts by weight, 0.05 parts by weight parts by weight to 0.1 parts by weight, 0.05 parts by weight parts by weight to 0.09 parts by weight, 0.05 parts by weight parts by weight to 0.08 parts by weight, 0.05 parts by weight to 0.07 parts by weight, 0.06 parts by weight to 0.115 parts by weight, 0.06 parts by weight to 0.11 parts by weight, 0.06 parts by weight to 0.105 parts by weight, 0.06 parts by weight to 0.1 parts by weight, 0.06 parts by weight to 0.09 parts by weight, 0.06 to 0.08 parts by weight, or 0.06 to 0.07 parts by weight of glycerol per 1 part by weight of the aqueous solution containing chitosan of the first agent.

In an embodiment, in the composition for preparing the multi-crosslinked temperature-sensitive hydrogel composition, the second agent may include 0.025 parts by weight to 0.120 parts by weight, 0.062 parts by weight to 0.2 parts by weight, 0.065 parts by weight to 0.2 parts by weight, 0.07 parts by weight to 0.2 parts by weight, 0.08 parts by weight to 0.2 parts by weight, 0.09 parts by weight to 0.2 parts by weight, 0.1 parts by weight to 0.2 parts by weight, 0.11 parts by weight to 0.2 parts by weight, 0.12 parts by weight to 0.2 parts by weight, 0.062 parts by weight to 0.19 parts by weight, 0.065 parts by weight to 0.19 parts by weight, 0.07 parts by weight to 0.19 parts by weight, 0.08 parts by weight to 0.19 parts by weight, 0.09 parts by weight to 0.19 parts by weight, 0.1 parts by weight to 0.19 parts by weight, 0.11 parts by weight to 0.19 parts by weight, 0.12 parts by weight to 0.12 parts by weight to 0.19 parts by weight, 0.07 parts by weight to 0.18 parts by weight, 0.08 parts by weight to 0.18 parts by weight, 0.09 parts by weight to 0.18 parts by weight, 0.1 parts by weight to 0.18 parts by weight, 0.11 parts by weight to 0.18 parts by weight, 0.12 parts by weight to 0.18 parts by weight, 0.07 parts by weight to 0.17 parts by weight, 0.08 parts by weight to 0.17 parts by weight, 0.09 parts by weight to 0.17 parts by weight, 0.1 parts by weight to 0.17 parts by weight, 0.11 parts by weight to 0.17 parts by weight, 0.12 parts by weight to 0.17 parts by weight, 0.07 parts by weight to 0.16 parts by weight, 0.08 parts by weight to 0.16 parts by weight, 0.09 parts by weight to 0.16 parts by weight, 0.1 parts by weight to 0.16 parts by weight, 0.11 parts by weight to 0.16 parts by weight, 0.12 parts by weight to 0.16 parts by weight, 0.07 parts by weight to 0.15 parts by weight, 0.08 parts by weight to 0.15 parts by weight, 0.09 parts by weight to 0.15 parts by weight, 0.1 parts by weight to 0.15 parts by weight, 0.11 parts by weight to 0.15 parts by weight, 0.12 parts by weight to 0.15 parts by weight, 0.07 parts by weight to 0.14 parts by weight, 0.08 parts by weight to 0.14 parts by weight, 0.09 parts by weight to 0.14 parts by weight, 0.1 parts by weight to 0.14 parts by weight, 0.11 parts by weight to 0.14 parts by weight, 0.12 parts by weight to 0.14 parts by weight, 0.07 parts by weight to 0.13 parts by weight, 0.08 parts by weight to 0.13 parts by weight, 0.09 parts by weight to 0.13 parts by weight, 0.1 parts by weight to 0.13 parts by weight, 0.11 parts by weight to 0.13 parts by weight, 0.12 parts by weight to 0.13 parts by weight, or 0.115 parts by weight to 0.125 parts by weight of glycerol per 1 part by weight of the aqueous solution containing chitosan of the first agent.

The second agent composition may further include a decellularized matrix.

In an embodiment, when the weight ratio of the aqueous solution containing chitosan and the aqueous solution containing phosphate ions in the total volume of the first agent is 1:0.11 or more but less than 0.13, the elasticity of the biomaterial composition may increase with an increase in a glycerol content of the second agent. For example, when the weight ratio of the aqueous solution containing chitosan and the aqueous solution containing phosphate ions is in the range of 1:0.12 or more but less than 0.013, an increase in the glycerol content in the range of 20 to 500 Pas, depending on the level of glycerol in the second agent composition added thereto, may grant strengthened elasticity to the biomaterial composition.

In another embodiment, when the weight ratio of the aqueous solution containing chitosan to the aqueous solution containing phosphate ions in the total volume of the first agent is in the range of 1:0.16 to 0.18, the elasticity of the biomaterial composition may rather decrease with an increase in the content of glycerol in the second agent. For example, when the volume ratio of the aqueous solution containing chitosan and the aqueous solution containing phosphate ions is in the range of 1:0.16 to 0.018, a decrease in the glycerol content in the range of 3000 to 7000 Pa's, depending on the level of glycerol in the second agent composition added thereto, may grant strengthened elasticity to the biomaterial composition. In other words, adjustment of the weight ratio between the first agent and second agent may allow for a secondary adjustment of the elasticity of the biomaterial composition.

The weight ratio of the first agent and second agent may be 1:10 to 10000:1, for example, the weight ratio of the first agent and second agent may be, for example, 1:5 to 10000:1, 1:3 to 10000:1, or 1:2 to 10000:1. For example, the weight ratio of the first agent and second agent may be from 2:1 to 10000:1. In this case, when the weight ratio is less than the above range, there is a problem that gelation does not occur or the strength level of the formed hydrogel is very weak, and when the weight ratio is above the above range, there is a problem that gelation does not occur or the strength level of the formed hydrogel is very weak. If the ratio of the first agent is high and the concentration of the phosphate group is excessive, gelation within the first agent will proceed even at room temperature, and the unique characteristic of the temperature-sensitive filler, which is injected in liquid form and gelation within the body, will be lost, if the ratio of the second agent is high and the concentration of glycerol is excessive, there is a problem in that the concentration of the solution becomes diluted and gelation does not occur in the body or at high temperatures, or the physical properties of the formed hydrogel become very weak.

In an example, the composition may include 70 wt % to 90 wt %, 75 wt % to 90 wt %, 80 wt % to 90 wt %, 81 wt % to 90 wt %, 82 wt % to 90 wt %, 83 wt % to 90 wt %, 84 wt % to 90 wt %, 85 wt % to 90 wt %, 86 wt % to 90 wt %, 70 wt % to 89 wt %, 75 wt % to 89 wt %, 80 wt % to 89 wt %, 81 wt % to 89 wt %, 82 wt % to 89 wt %, 83 wt % to 89 wt %, 84 wt % to 89 wt %, 85 wt % to 89 wt %, 86 wt % to 89 wt %, 70 wt % to 88 wt %, 75 wt % to 88 wt %, 82 wt % to 88 wt %, 83 wt % to 88 wt %, 84 wt % to 88 wt %, 85 wt % to 88 wt %, 86 wt % to 88 wt %, 70 wt % to 87 wt %, 75 wt % to 87 wt %, 80 wt % to 87 wt %, 81 wt % to 87 wt %, 82 wt % to 87 wt %, 83 wt % to 87 wt %, 84 wt % to 87 wt %, 85 wt % to 87 wt %, or 86 wt % to 87 wt %, of an aqueous solution containing chitosan relative wt % to the total weight.

In an example, the composition may include 1 wt % to 20 wt %, 5 wt % to 20 wt %, 5 wt % to 17 wt %, 7 wt % to 17 wt %, 8 wt % to 17 wt %, 9 wt % to 17 wt %, 10 wt % to 17 wt %, 5 wt % to 15 wt %, 7 wt % to 15 wt %, 8 wt % to 15 wt %, 9 wt % to 15 wt %, 10 wt % to 15 wt %, 7 wt % to 14.5 wt %, 7 wt % to 13 wt %, 8 wt % to 13 wt %, 9 wt % to 13 wt %, 10 wt % to 13 wt %, 7 wt % to 12 wt %, 8 wt % to 12 wt %, 9 wt % to 12 wt %, 10 wt % to 14.5 wt %, 10 wt % to 12 wt %, 10 wt % to 11 wt %, or 10.5 wt % to 12 wt % of a phosphoric acid solution based on the total weight.

In an example, the composition may include 0.1 wt % to 20 wt %, 0.1 wt % to 15 wt %, 0.5 wt % to 15 wt %, 1 wt % to 15 wt %, 0.5 wt % to 13 wt %, 1 wt % to 13 wt %, 1 wt % to 12 wt %, 1 wt % to 11 wt %, 1 wt % to 10 wt %, 1.5 wt % to 15 wt %, 1.5 wt % to 13 wt %, 1.5 wt % to 12 wt %, 1.5 wt % to 11 wt %, 2 wt % to 15 wt %, 2 wt % to 13 wt %, 2 wt % to 12 wt %, 2 wt % to 11 wt %, or 2 wt % to 10 wt % of glycerol relative to the total weight.

In an embodiment, the composition for preparing the temperature-sensitive hydrogel composition may further include a third agent in a liquid formulation containing a basic aqueous solution for changing strength of the hydrogel composition. In an example, further including a third agent in the hydrogel composition may change the strength of the hydrogel composition.

The “basic aqueous solution” included in the third agent herein may contribute to strengthening the strength of the formed temperature-sensitive hydrogel and may be provided, as an example, in a form of an aqueous solution containing basic ions. The basic aqueous solution may include, for example, one type or more selected from the group consisting of ammonia, lithium hydroxide, sodium hydroxide, potassium hydroxide, calcium hydroxide, magnesium hydroxide, zinc hydroxide, cesium hydroxide, barium hydroxide, rubidium hydroxide, iron(II) hydroxide, iron(III) hydroxide, aluminum hydroxide, methylamine, ethylamine, n-propylamine, n-butylamine, calcium carbonate, potassium bicarbonate, sodium carbonate, and sodium bicarbonate solutions, but the basic ion may be extended and applied without limitation as long as it is a basic ion.

In an embodiment, the first agent and second agent may be isolated in separate spaces within the container. Therefore, under conditions before use, the first agent containing chitosan and phosphate ions and the second agent containing glycerol may each be maintained and stored in a liquid formulation.

In an embodiment, the first agent and second agent may be subjected to a sequential mixing and stabilization process to form a temperature-sensitive hydrogel that undergoes gelation under in vivo condition. As shown in FIG. 1, the first agent may be subjected to a mixing and stabilization process between an aqueous solution containing chitosan and phosphate ions to form ionic bonds between the amine groups of chitosan and phosphate ions, which may be involved in determining the strength level of the hydrogel. Thereafter, mixing the stabilized first agent with the second agent containing glycerol form a covalent bond between the remaining amine group of chitosan and glycerol, and this process may be involved in determining the viscoelasticity of the hydrogel.

In an embodiment, the first agent and second agent may be mixed. Therefore, under conditions before use, the first agent containing chitosan and phosphate ions and the second agent containing glycerol may each be maintained and stored in a liquid formulation or a gel formulation in a mixed state and, if desired, may be frozen for long-term storage.

In an embodiment, the first agent and second agent may be subjected to a sequential mixing and stabilization process to form a temperature-sensitive hydrogel that undergoes gelation in vitro.

Another aspect provides a method of preparing the temperature-sensitive hydrogel composition, including a phase of mixing a solution including chitosan ions and the aqueous solution containing phosphate ions to prepare the first agent in the liquid formulation, and stabilizing the first agent in the liquid formulation.

Another aspect provides a method of preparing a temperature-sensitive hydrogel composition including, after the phase of stabilizing, further including the phase of mixing the stabilized first agent in the liquid formulation with the second agent of the liquid formulation containing glycerol.

Another aspect provides a method of preparing a temperature-sensitive hydrogel composition, further including the phase of multi-crosslinking the liquid formulation mixture of the first agent and second agent under an in vivo condition.

Another aspect provides a method of preparing a temperature-sensitive hydrogel composition, further including the phase of further mixing a third agent including a basic aqueous solution to change the strength of the hydrogel composition.

Another aspect provides a method of administering a temperature-sensitive hydrogel composition, including the phase of preparing a first agent of a liquid formulation by mixing a solution containing chitosan ions and an aqueous solution including phosphate ions; stabilizing the first agent in the liquid formulation; mixing the first agent of the stabilized liquid formulation with a second agent of the liquid formulation including glycerol; and injecting the mixed liquid formulation into the skin of a subject.

Since the method of preparing the temperature-sensitive hydrogel composition or the method of administering the temperature-sensitive hydrogel composition includes or utilizes the composition for preparing the temperature-sensitive hydrogel composition as described above, any content common between the two will be omitted.

According to an aspect, the temperature-sensitive hydrogel prepared by the above methods not only provide the practitioner with convenience for indication application, but also may retain their form for a longer period of time under in vivo conditions compared to an existing hydrogel composition, and the physical property of the hydrogel, specifically elasticity and strength, may be easily adjusted to provide the hydrogel that conform to the form and property of various forms of tissue.

### Advantageous Effects

According to an aspect, a hydrogel composition in a liquid formulation containing a mixture of a first agent and second agent form a temperature-sensitive hydrogel that undergoes gelation under an in vivo condition after injection, thereby providing convenience in distribution and hydrogel procedures, and may maintain its shape for a long time under the in vivo condition compared to an existing hydrogel composition.

Additionally, according to a composition according to an aspect, the strength of the formed hydrogel composition may be easily changed by applying a basic aqueous solution such as an aqueous sodium hydroxide solution, etc. In an aspect, the elasticity and strength of the hydrogel composition may be changed according to an indication, thereby providing a hydrogel that conform to the form and property of various tissue.

### DESCRIPTION OF DRAWINGS

FIG. 1A is a diagram illustrating bond formation over time in a temperature-sensitive hydrogel according to an example.

FIG. 1B is a diagram schematically illustrating a change in a physical property according to a change in temperature of a temperature-sensitive hydrogel according to an example.

FIG. 2 is a diagram illustrating a mixing process of a first agent and second agent in liquid form, according to an example.

FIG. 3 shows a result of evaluating a change in viscosity over time in a first agent including chitosan and phosphate ions according to an example.

FIG. 4 is a diagram illustrating a degree of gelation according to a ratio of components of the first agent and second agent of Example 2.2.1 to Example 2.2.9.

FIG. 5A illustrates a gelation viscosity according to a component ratio of the first agent and second agent of Example 2.2.1 (G1) to Example 2.2.3 (G3).

FIG. 5B illustrates the gelation viscosity according to the component ratio of the first agent and second agent in Example 2.2.4 (G4) to Example 2.2.6 (G6).

FIG. 5C illustrates the gelation viscosity according to the component ratio of the first agent and second agent in Example 2.2.7 (G7) to Example 2.2.9 (G9).

FIG. 6A is a diagram illustrating a complex viscosity according to an angular frequency of Example 2.2.1 (G1) to Example 2.2.9 (G9).

FIG. 6B is a diagram illustrating a loss modulus of Example 2.2.1 (G1) to Example 2.2.9 (G9).

FIG. 6C is a diagram illustrating a storage modulus of Example 2.2.1 (G1) to Example 2.2.9 (G9).

FIG. 7A is a diagram depicting a photograph of a hydrogel formed at 37° C. after mixing the first agent and second agent of the liquid formulation.

FIG. 7B is a diagram comparing a compressive strength of the hydrogel formed at 37° C. after mixing the first agent and second agent of the liquid formulation at weeks 1, 2, 3, and 4.

FIG. 8A is a diagram comparing a recovery rate after compression according to a pressure of the hydrogel formed at 37° C. mixing the first agent and second agent of the liquid formulation.

FIG. 8B is a graph illustrating the quantification of the recovery rate according to the pressure of the hydrogel formed at 37° C. after mixing the first agent and second agent of the liquid formulation.

FIG. 8C is a diagram comparing a deformation after compression of the hydrogel formed under 37° C. condition after mixing the first agent and second agent of the liquid formulations of Example 2.2.7 (G7) to Example 2.2.9 (G9).

FIG. 9 illustrates the viscosity before and after crosslinking for 1 hour by adding the first agent, the second agent, and a third agent including NaOH.

FIG. 10A shows a result of injecting the first agent and second agent of the liquid formulation according to an example into a mouse and determining the shape of the hydrogel formed intradermally in the mouse through the naked eye or a microscope.

FIG. 10B is a diagram comparing an E-modulus over time and an E-modulus within the mouse skin after injecting the first agent and second agent of the liquid formulation according to an example into the mouse.

FIG. 10c is a diagram comparing a degree of the E-modulus over time and compressive strength in the mouse skin after injection of the first agent and second agent of the liquid formulation according to an example into a mouse.

FIG. 11A is a diagram illustrating an observation of an appearance of the mouse one week after injection of the first agent and second agent of the liquid formulation according to an example into a mouse.

FIG. 11B is a diagram illustrating the hydrogel taken out of the mouse and checked at the end of one week after injecting a mixture of the first agent and second agent of the liquid formulation according to an example into the dermis of the mouse.

FIG. 12A shows the results of injecting PBS into mice and then checking for an immune response in the body.

FIG. 12B shows the results of confirming whether there is an immune response in the body caused by chitosan filler (hydrogel) formed intradermally in a mouse after injecting the first agent and second agent of the liquid formulation according to an example.

FIG. 12C is a diagram comparing a CD68 staining area of PBS and chitosan filler (hydrogel) at days 3, 7, and 14.

FIG. 12D is a diagram comparing a CD206 staining area of PBS and chitosan filler (hydrogel) at days 3, 7, and 14.

FIG. 13 is a diagram confirming an increase in the physical property due to simultaneous temperature and body fluid-responsive gelation.

FIG. 14A is a diagram visually confirming the hydrogel sensitized with body fluid after temperature-sensitive gelation in vitro.

FIG. 14b is a diagram comparing the E-modulus of the hydrogel formed intradermally in the mouse after mixing the first agent and second agent of the liquid formulation (separate type) and the hydrogel formed in vitro after mixing the first agent and second agent of the liquid formulation and gelating in vivo, and the hydrogel formed intradermally in the mouse after fluid-sensitive gelation (integral type).

FIG. 14C is a diagram comparing the compressive strength of the hydrogel formed intradermally in the mouse after mixing the first agent and second agent of the liquid formulation (separate type) and a hydrogel formed in vitro after mixing the first agent and second agent of the liquid formulation and gelating in vivo, and the hydrogel formed intradermally in the mouse after fluid-sensitive gelation (integral type).

FIG. 15 is a diagram comparing the physical property of the hydrogel formed intradermally in the mouse after mixing the first agent and second agent of the liquid formulation (separate type) and the hydrogel formed in vitro after mixing the first agent and second agent of the liquid formulation and gelating in vivo, and the hydrogel formed intradermally in the mouse after fluid-sensitive gelation (integral type).

FIG. 16 shows a results of comparing a degradation rate according to a cross-linking agent concentration of the hydrogel formed intradermally in the mouse after mixing the first agent and second agent of the liquid formulation according to an example.

FIG. 17 is a diagram illustrating an observation of an appearance of the hydrogel before and after injection into a mouse after mixing the first agent and second agent of the liquid formulation including the decellularized material according to an example, and the mouse injected with the first agent and second agent of the mixed liquid formulation.

FIG. 18 shows a results of quantifying the elasticity of the hydrogel before and after injection into the body as confirmed in FIG. 17 above.

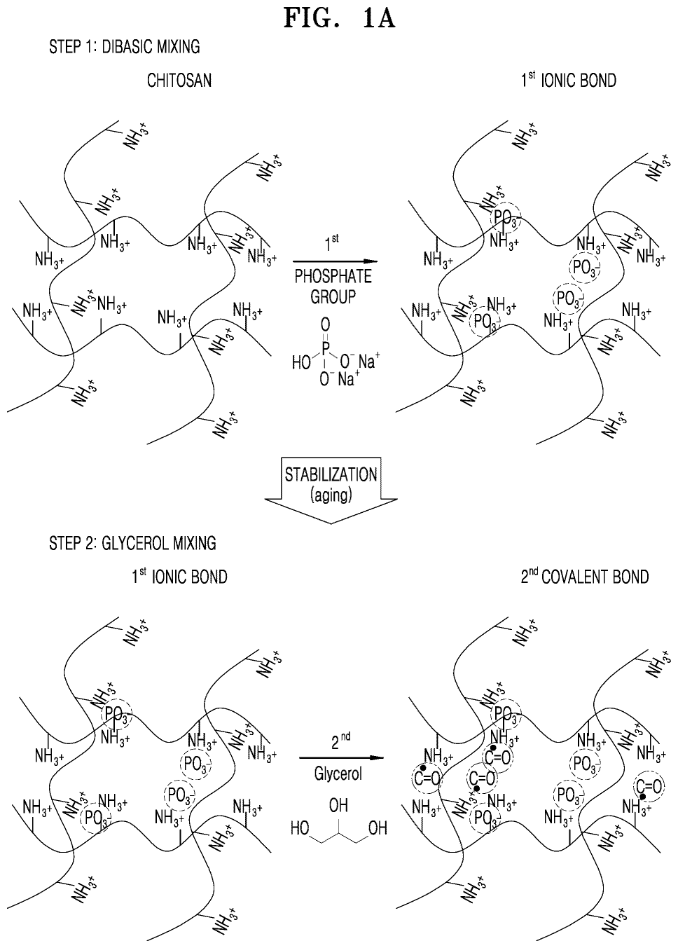

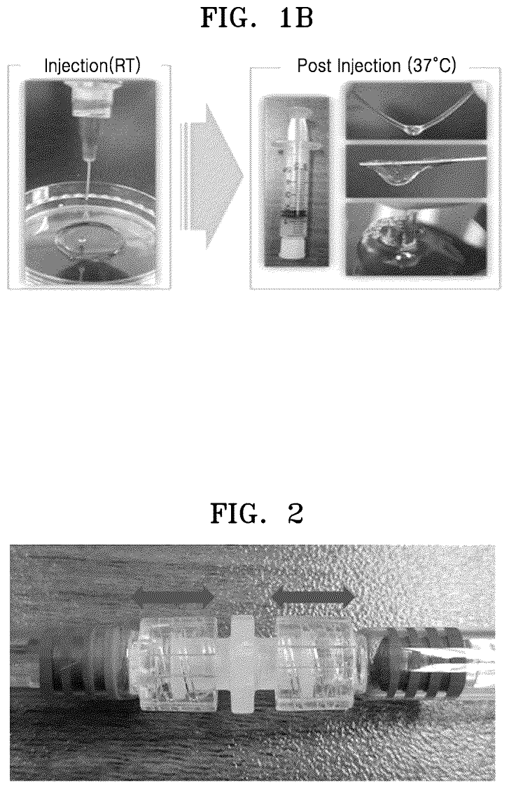

### BEST MODE

Hereinafter, preferred examples are presented to aid understanding of the present disclosure. However, the following examples are provided only to aid understanding of the present disclosure, and the present disclosure is not limited by the following examples.

### EXAMPLE

### Example 1. Preparation of Composition for Preparing Temperature-Sensitive Hydrogel

### Example 1.1. Preparation of First Agent Including Chitosan and Phosphate Ions

In the present example, 500 ml of 1N HCl aqueous solution was prepared by mixing 44.05 ml of 35 to 37% HCl solution with 455.95 ml of distilled water. In addition, 125 g of chitosan powder was added to 4500 ml of distilled water and stirred to disperse the chitosan powder. 500 ml of 1N HCl aqueous solution was added here, and mixed for about 1 hour in a water bath at 60° C. to prepare an aqueous solution containing chitosan.

Meanwhile, 98.49 g of sodium phosphate dibasic (Na₂HPO₄) was completely dissolved in 550 ml of distilled water, and then filtered through a 0.45 μm filter once to prepare a sodium phosphate dibasic solution. Then, under continuous stirring, 5000 ml of the aqueous solution containing the above chitosan was titrated with 550 ml of the above sodium phosphate dibasic solution to prepare a first agent of a liquid formulation according to an example. Then, 1 ml syringes were filled with 1.1 ml of the above mixed solution, autoclaved, and stored at room temperature.

### Example 1.2. Preparation of Second Agent Including Glycerol

In the present example, 100% glycerol was used to prepare a second agent.

### Example 2. Preparation of Liquid Formulation of Composition

### Example 2.1. Preparation of Composition Including First Agent

In the present example, the first agent in the liquid formulation prepared in Example 1 above was used to prepare a hydrogel composition forming a gel formulation. After thoroughly mixing the first agent, a hydrogel composition in a liquid formulation was prepared.

### Example 2.2. Preparation of Composition Including First Agent and Second Agent

In the present example, the first agent and second agent of the liquid formulation prepared in Example 1 were used to prepare the hydrogel composition forming the gel formulation. An example of the preparation process of a hydrogel composition in liquid form at room temperature is shown in FIG. 2. Specifically, the sealing cap of the syringe containing the first agent or second agent, respectively, were opened, and the syringes were connected using a connector. Thereafter, the first agent and second agent were sufficiently mixed by moving the pushers of both syringes, and then the mixture was moved into one syringe to prepare a hydrogel composition in a liquid formulation.

The weight ratio of the aqueous solution containing chitosan, sodium phosphate dibasic solution, and glycerol included in the first agent and second agent is shown in Table 1 below.

TABLE 1

| | Aqueous solution | Phosphoric acid | |
|---|---|---|---|
| Example | containing chitosan | solution | Glycerol |
| 2.2.1 (G1) | 1 | 0.126 | 0.024 |
| 2.2.2 (G2) | | | 0.061 |
| 2.2.3 (G3) | | | 0.122 |
| 2.2.4 (G4) | | 0.136 | 0.024 |
| 2.2.5 (G5) | | | 0.061 |
| 2.2.6 (G6) | | | 0.122 |
| 2.2.7 (G7) | | 0.169 | 0.024 |
| 2.2.8 (G8) | | | 0.061 |
| 2.2.9 (G9) | | | 0.122 |

(Unit: parts by weight)

### Experimental Example

### Experimental Example 1. Evaluation of Viscosity of First Agent Including Chitosan and Phosphate Ions

In the present experimental example, a viscosity of the first agent in the liquid formulation was evaluated over time to derive a stabilization period of the first agent that may induce a desired change in a physical property before mixing with the second agent. The first agent in the liquid formulation prepared above was sealed and stored at room temperature for 1 week, 2 weeks, 3 weeks, and 4 weeks, respectively, and the viscosity change was evaluated. A total of 16 ml of the first agent was evaluated for viscosity using a Brookfield viscometer DV2TLV, Small sample adapter-spindle, at room temperature, with a total of 10 points measured at 30-second intervals for 5 minutes (Multipoint method).

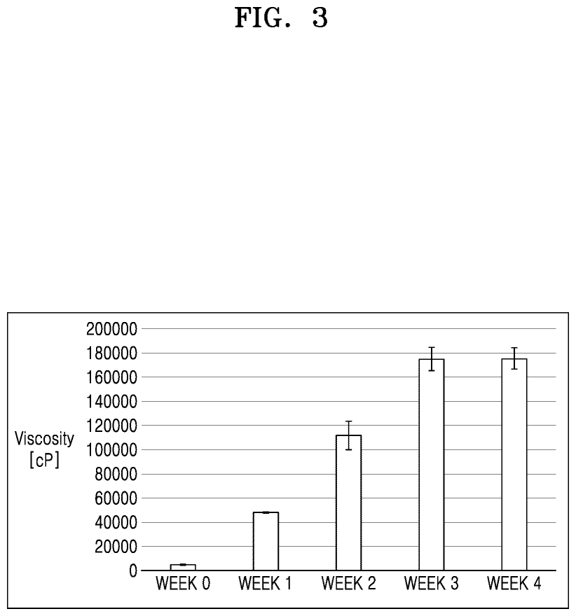

FIG. 3 shows the results of evaluating a change in viscosity over time of the first agent including chitosan and phosphate ions. As shown in FIG. 3, a viscosity property of the liquid formulation of the first agent were obviously different immediately after an autoclaving process and after stabilization at room temperature for a period of time, and the physical property tended to stabilize to some extent after about several days at room temperature after the first agent was prepared.

### Experimental Example 2. Performance Evaluation of Temperature-Sensitive Hydrogel

In the present experimental example, an injection force of the liquid formulation before gelation after mixing the first agent and second agent of the liquid formulation, a viscoelasticity of the hydrogel formed at 37° C., a degree of gelation according to a ratio of the component of the first agent and second agent, and the viscosity according to the gelation were evaluated. In the injection force evaluation, the first agent in the liquid formulation was stored at room temperature for 1 week, 2 weeks, 3 weeks, or 4 weeks, and then mixed with the second agent of the liquid formulation. Afterwards, a 26G needle was connected and the injection force was evaluated using AND MCT-2150 at room temperature and a test speed of 10 mm/min.

In addition, in the viscoelasticity evaluation, the first agent in the liquid formulation was stored at room temperature for 1 week, 2 weeks, 3 weeks, or 4 weeks, and then mixed with the second agent of the liquid formulation. Afterwards, the filler composition of the liquid formulation was then stored in a 37° C. incubator for 24 hours, and the viscoelasticity of the resulting tissue repair hydrogel was measured at 25° C., 0.628 to 198 rad/s, using a rotational rheometer (TA instrument Ltd., ARES-G2). Meanwhile, a comparison group used the filler composition of Allaergan, which is currently on the market.

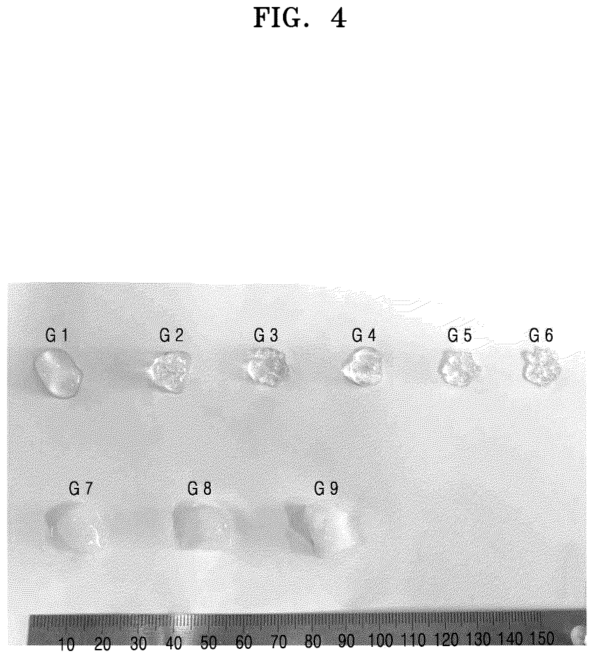

FIG. 4 is a diagram illustrating a degree of gelation according to a ratio of components of the first agent and second agent of Example 2.2.1 to Example 2.2.9. In the present experimental example, the effect of the content of sodium phosphate dibasic and/or glycerol on gel formation of the hydrogel composition was examined. Based on the ratio of each component in Example 2.2.1 to Example 2.2.9 above, the first agent including chitosan and sodium phosphate dibasic solution, and the second agent including glycerol were mixed to prepare a total of 9 hydrogel compositions in liquid formulation. Thereafter, the hydrogel composition for tissue repair was stored in an incubator at 37 ºC for about 24 hours, and then the progress of gelation was evaluated.

As shown in FIG. 4, it was confirmed that the physical property of the hydrogel may be changed depending on the ratio of sodium phosphate dibasic and glycerol, and according to such changes in the physical property, gelation progresses to a level where the hydrogel may be used to replace the tissue.

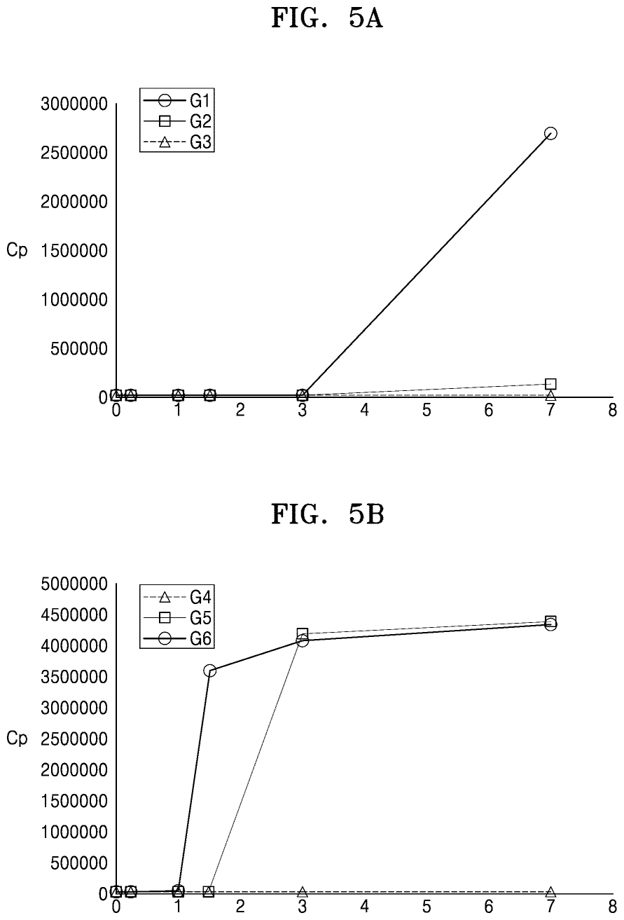

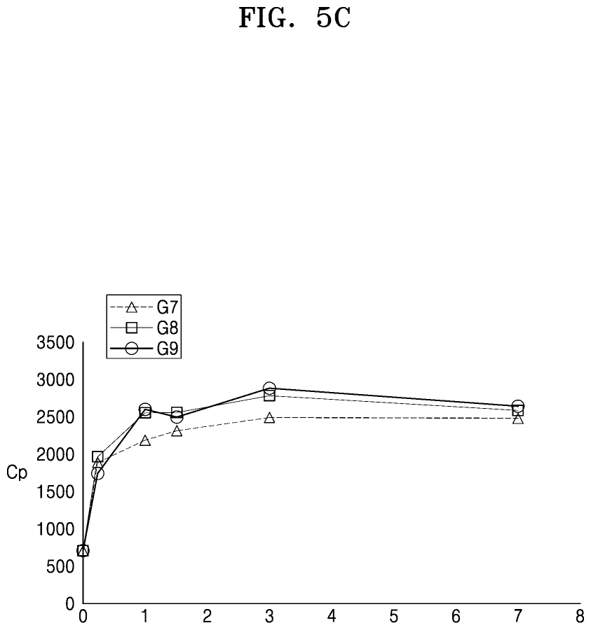

FIG. 5 is a diagram illustrating the viscosity upon gelation according to a ratio of components of the first agent and second agent of Example 2.2.1 to Example 2.2.9. As shown in FIG. 5, the gelation time was faster as the ratio of diphosphate and glycerol increased. Specifically, the increase in viscosity value due to gelation in 2.2.5 and 2.2.6 was the largest, and the viscosity increase in 2.2.6 appeared earlier compared to 2.2.5.

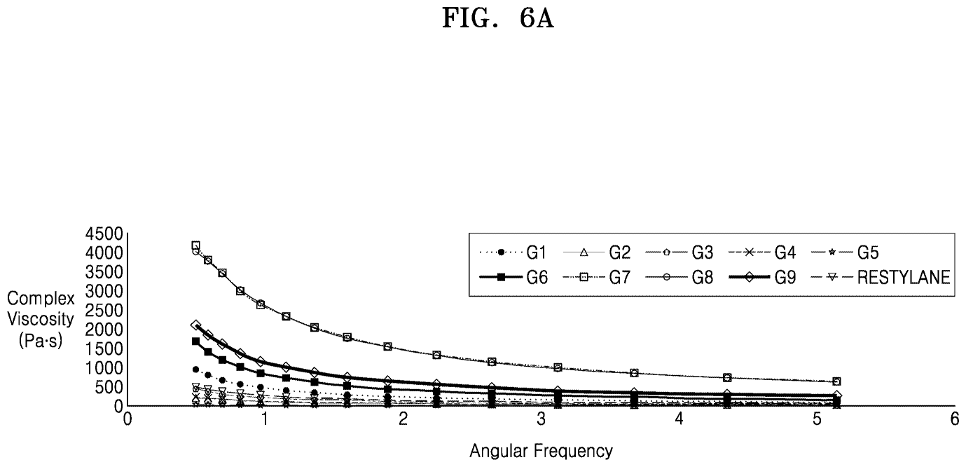

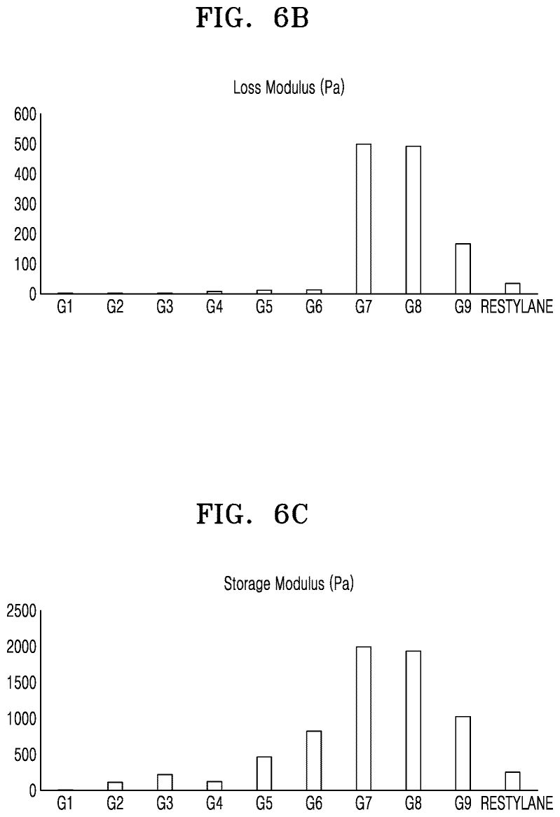

Additionally, Table 2 and FIG. 6 show the results of confirming changes in the physical property according to angular frequency in Example 2.2.1 to Example 2.2.9. FIG. 6A is a diagram illustrating a complex viscosity according to an angular frequency of Example 2.2.1 to Example 2.2.9. FIG. 6B is a diagram illustrating a loss modulus of Example 2.2.1 to Example 2.2.9. FIG. 6C is a diagram illustrating a storage modulus of Example 2.2.1 to Example 2.2.9. As shown in FIG. 6A to FIG. 6C, it was confirmed that the elasticity of the generated gelling composition may be changed by adjusting the ratio of chitosan, sodium phosphate dibasic, and glycerol included in first agent and second agent.

TABLE 2

| | 0.5 rad/s | | | | | | | | | |
|---|---|---|---|---|---|---|---|---|---|---|
| | | | | | | | | | | Crosslinked |
| | | | | | | | | | | hyaluronic |
| | G1 | G2 | G3 | G4 | G5 | G6 | G7 | G8 | G9 | acid |
| Complex | 25.634 | 202.38 | 432.94 | 231.88 | 950.86 | 1670.9 | 4200 | 4052.3 | 2111.7 | 497.93 |
| viscosity | | | | | | | | | | |
| (Pa · s) | | | | | | | | | | |
| Storage | 12.285 | 101.18 | 216.47 | 115.72 | 475.32 | 835.35 | 2011.1 | 1964.1 | 1042.2 | 246.51 |
| modulus | | | | | | | | | | |
| (Pa) | | | | | | | | | | |
| Loss | 3.652 | 0.8764 | 1.2363 | 7.0733 | 10.54 | 13.411 | 503.123 | 497.68 | 169.34 | 34.885 |
| modulus | | | | | | | | | | |
| (Pa) | | | | | | | | | | |

### Experimental Example 3. Evaluation of Compressive Strength of Temperature-Sensitive Hydrogel

In the present experimental example, a compressive strength of the hydrogel formed at 37° C. was evaluated after mixing the first agent and second agent of the liquid formulation. The first agent in the liquid formulation was stored at room temperature for 1 week, 2 weeks, 3 weeks, or 4 weeks, and then mixed with the second agent of the liquid formulation. Thereafter, the liquid formulation of the hydrogel composition was stored in an incubator at 37° C. for 2 hours or 4 hours, respectively, and the compressive strength of the hydrogel formed accordingly was measured. The sample size was adjusted to 12 mm in diameter and 8.7 mm in length, and the compressive strength was evaluated using an AND MCT-2150 at room temperature and a test speed of 10 mm/min.

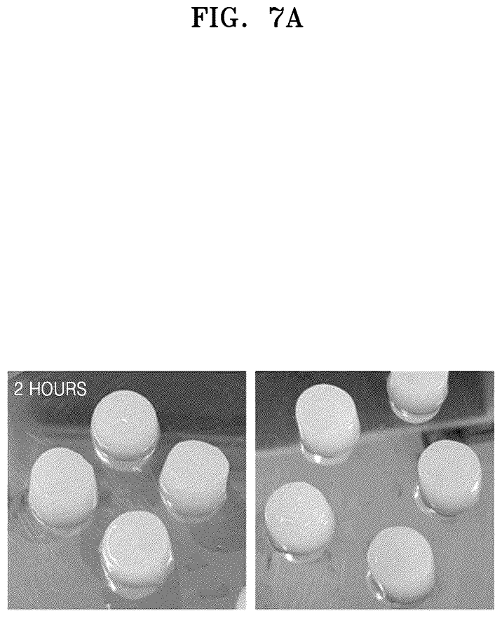

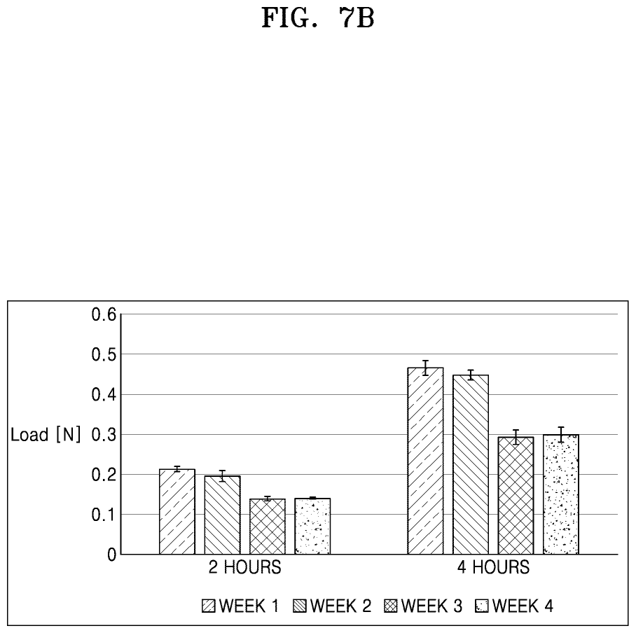

FIG. 7 shows the results of evaluating the compressive strength of the hydrogel formed at 37° C. after mixing the first agent and second agent of the liquid formulation. As shown in FIG. 7, it was confirmed that the hydrogel composition of the liquid formulation was converted to hydrogel form after about 2 hours at 37° C., and the compressive strength increased with time. These experimental results show that the hydrogel composition of the liquid formulation including a mixture of the first agent and second agent according to an aspect may be applied to prepare temperature-sensitive hydrogel that exist in an injectable or injectable form under room temperature conditions and undergo gelation under in vivo conditions after injection.

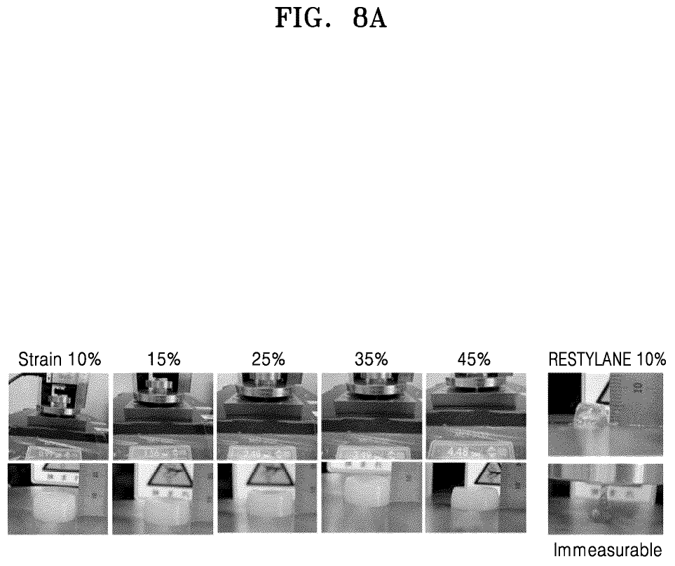

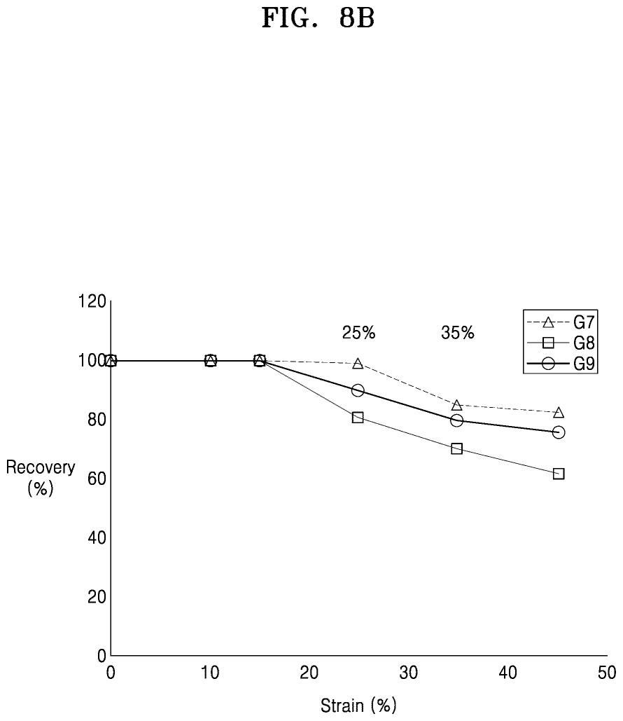

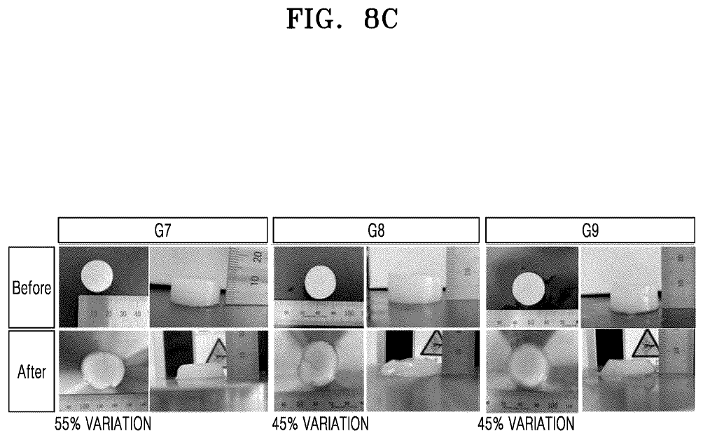

FIG. 8 shows the results of confirming the recovery rate after compression of the hydrogel formed under 37° C. conditions after mixing the first agent and second agent of the liquid formulation. To determine the recovery after compression, the hydrogel was compressed to a certain strain rate and the load was removed after 5 seconds. After removing the load, the height of the hydrogel was measured to calculate the recovery rate. As a result, as shown in FIG. 8A, the recovery rate was shown after applying a strain of 10% to 45%. FIG. 8B and FIG. 8C show the strain-dependent recovery of the hydrogel of Example 2.2.7 to Example 2.2.9. As shown in FIG. 8A to FIG. 8C, it was confirmed that the recovery rate of the generated gelling composition may be changed by adjusting the ratio of glycerol.

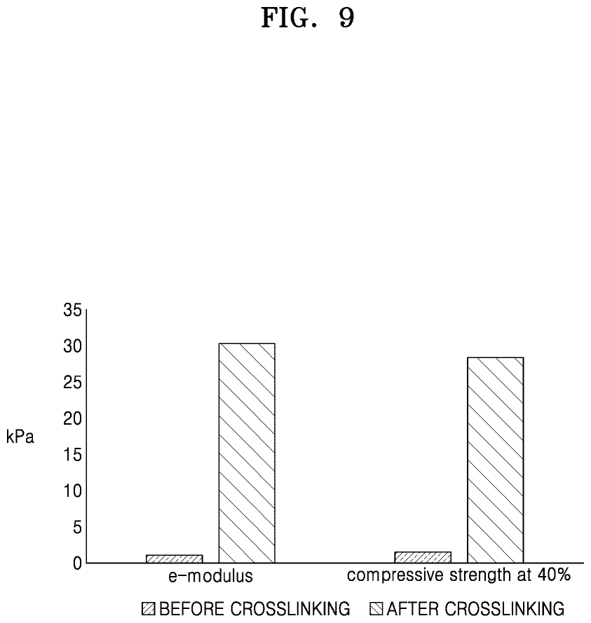

FIG. 9 shows the results of evaluating the compressive strength of a hydrogel that was tertiarily crosslinked by treating a hydrogel mixed with the first agent and second agent with a third agent including NaOH. In an example, the first agent (10 ml of 2.5% chitosan, 0.8 mL of 0.5 M dibasic) and the second agent (1.2 mL of glycerol) were mixed, homogenized, and printed. Afterwards, 30 mM NaOH was added for tertiary crosslinking for 1 hour. As a result, it was confirmed that the compressive strength of the hydrogel increased after tertiary crosslinking. These experimental results show that the hydrogel composition of a liquid formulation, which is a mixture of the first agent, second agent, and third agent according to an aspect, may be applied to a formulation that need to withstand greater pressure than the hydrogel mixture of the liquid formulation of the first agent and second agent alone under room temperature conditions.

### Experimental Example 4. Evaluation of Temperature-Sensitive Hydrogel Using Animal Model

### Experimental Example 4.1. Injection of Hydrogel in Liquid Formulation into the Body

In the present experimental example, an animal model was used to evaluate the change in the physical property and immune responses in the body of the temperature-sensitive hydrogel formed when injected into the body in liquid form.

Specifically, 0.1 ml of the liquid formulation of the hydrogel composition of Example 2.2.1 to Example 2.2.9, which was a mixture of the first agent and second agent, was transplanted intradermally in mice. Each after 2 weeks, 4 weeks, and 12 weeks, an autopsy was performed to observe the shape of the hydrogel, and tissue samples were prepared into paraffin blocks and sectioned for H&E and MT staining.

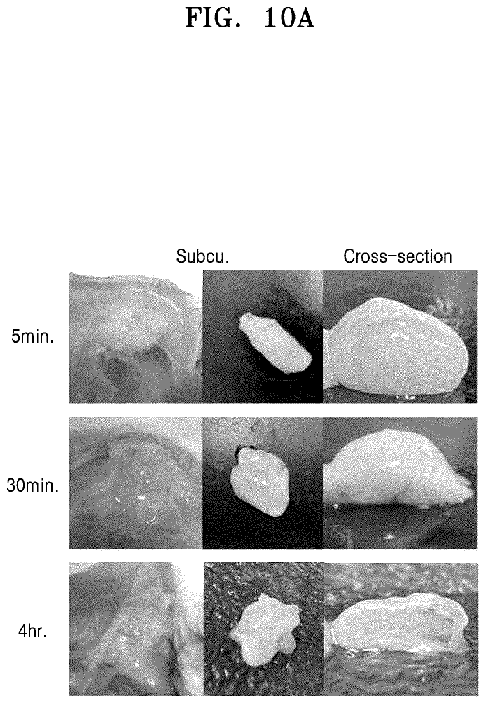

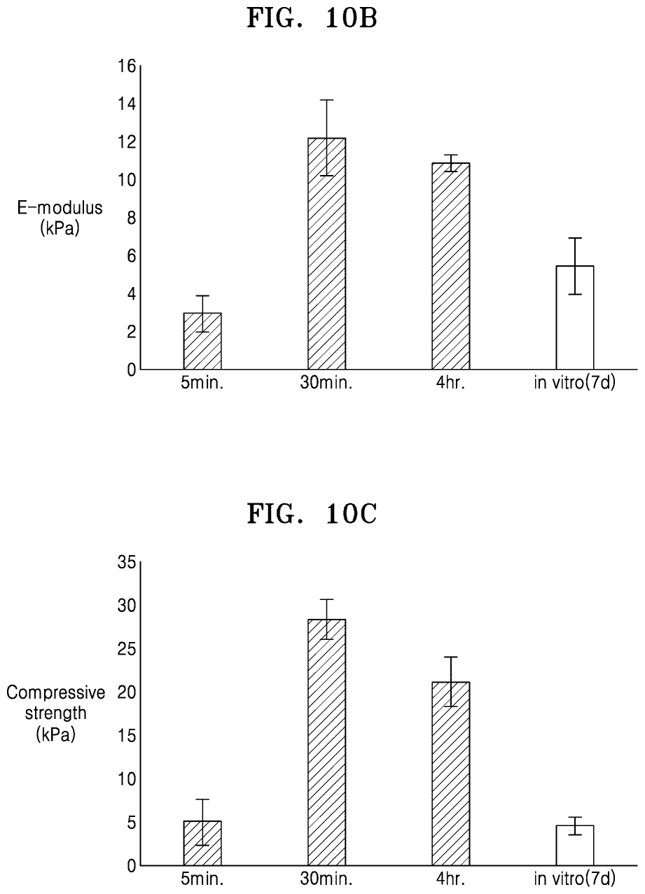

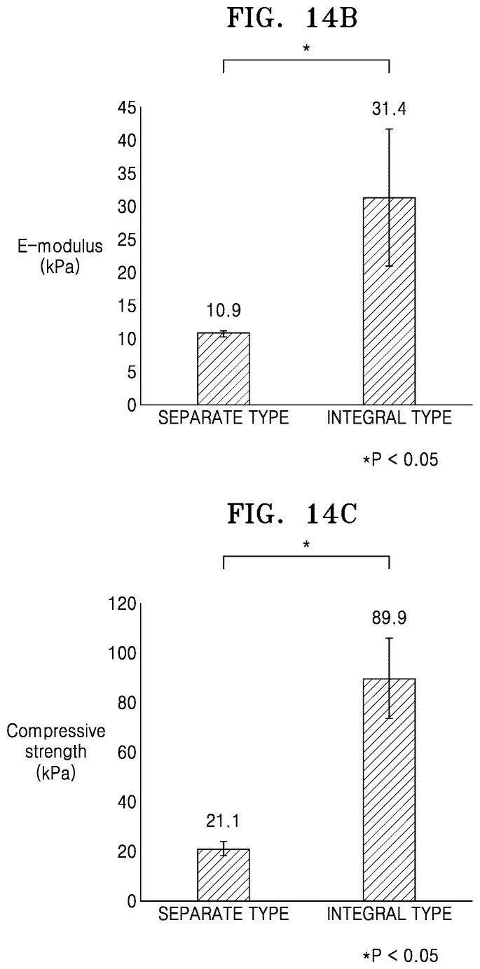

FIG. 10 shows a result of mixing the first agent and second agent of the liquid formulation according to an example into a mouse and determining the shape of the hydrogel formed intradermally in the mouse through the naked eye or a microscope. As shown in FIG. 14, the temperature-sensitive hydrogel according to an example was in a liquid form and gelation began immediately upon injection into the body, and gelation was completed within 30 minutes after the start.

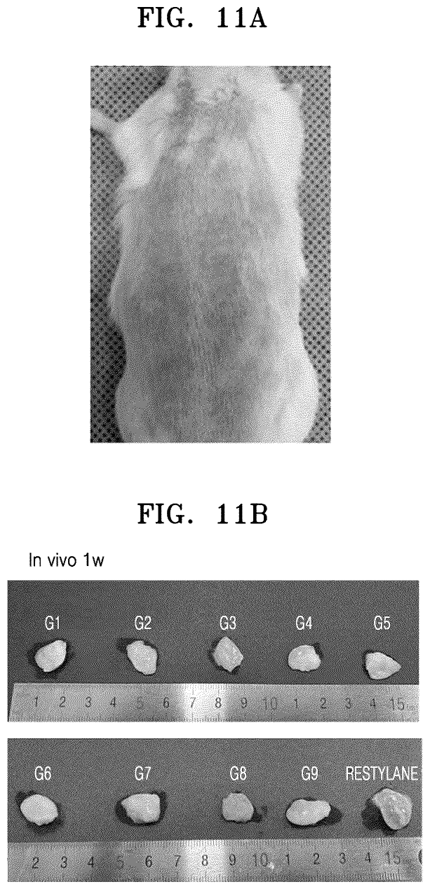

FIG. 11 shows the results of observing the hydrogel formed intradermally in the mouse one week after mixing the first agent and second agent of the liquid formulation according to an example.

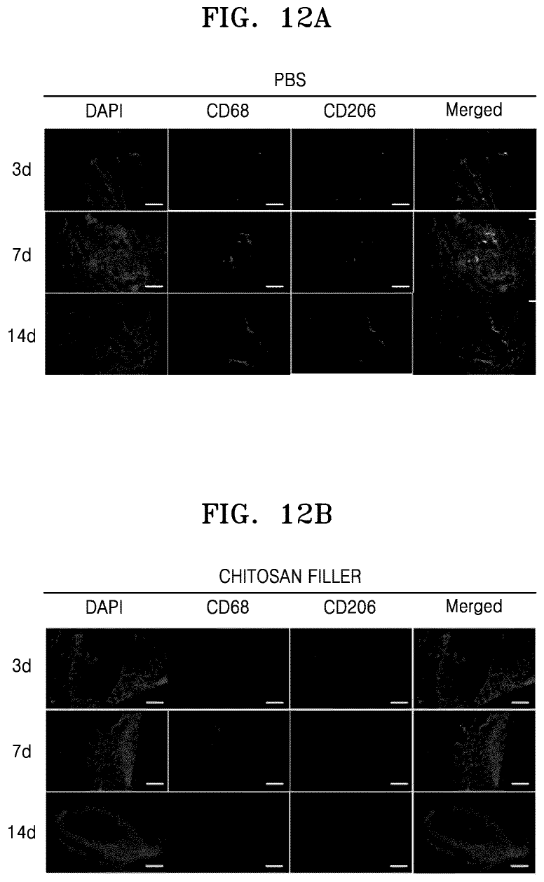

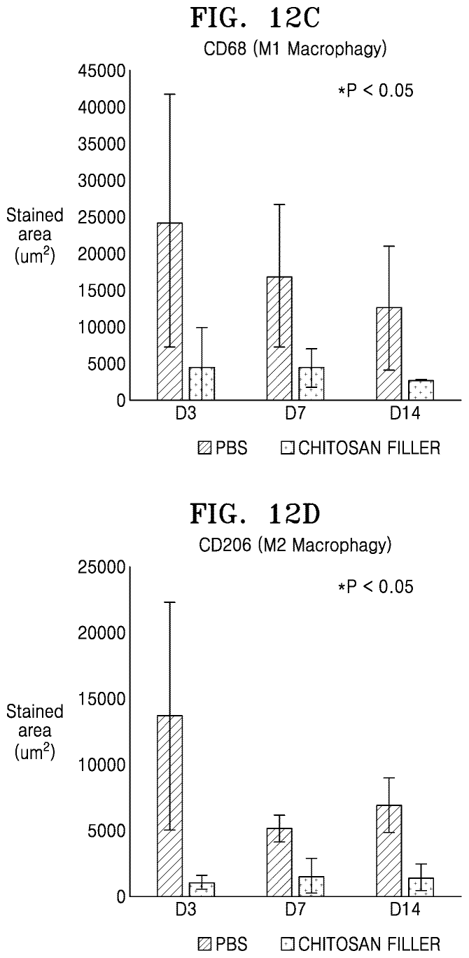

FIG. 12 shows the results of mixing the first agent and second agent of the liquid formulation according to an example, and then confirming whether there was an immune response in the body due to the hydrogel formed intradermally in the mouse. As shown in FIG. 12, no pathological findings including inflammatory reaction within the mouse skin were observed.

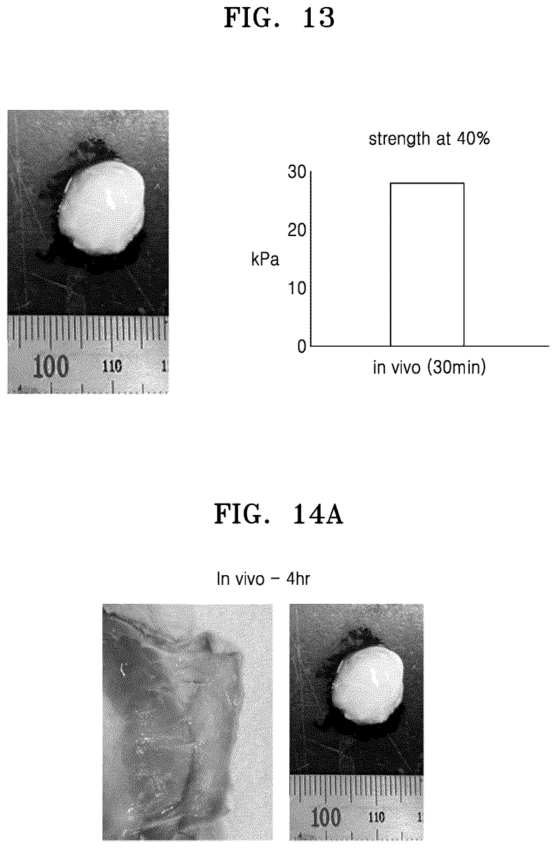

FIG. 13 is a diagram confirming an increase in the physical property due to simultaneous temperature and body fluid-responsive gelation. A liquid material mixed with first agent and second agent was compressed to a strain of 40% 30 minutes after being injected into the SD-rat body, and the physical property was confirmed. As a result, it was confirmed that the physical property of the temperature-sensitive hydrogel increased following gelation in response to temperature and body fluids.

### Experimental Example 4.2. Intracorporeal Injection after Gelation

In the present experimental example, an animal model was used to evaluate the change in the physical property of the formed temperature-sensitive hydrogel gelated in vitro by mixing the first agent and second agent and injected into the body.

Specifically, a mixture of the first agent and second agent mixed in the ratio of Example 2.2.1 to Example 2.2.9 was gelated in vitro and then transplanted into the skin of mice in an amount of 0.1 ml each. Each after 2 weeks, 4 weeks, and 12 weeks, an autopsy was performed to observe the shape of the hydrogel, and tissue samples were prepared into paraffin blocks and sectioned for H&E and MT staining samples were prepared into paraffin blocks and sectioned for H&E and MT staining.

FIG. 14 is a diagram confirming the increase in the physical property of the hydrogel when sensitized with body fluid after temperature-sensitive gelation in vitro. Specifically, the hydrogel composition for tissue repair in a liquid formulation, which was a mixture of the first agent and second agent, was stored in an incubator at 37° C. for about 24 hours to proceed with gelation. A gelated hydrogel was compressed to a strain of 40% 30 minutes after being injected into the SD-rat body, and the physical property was confirmed. As a result, it was confirmed that the physical property of the material were further improved according to body fluid response.

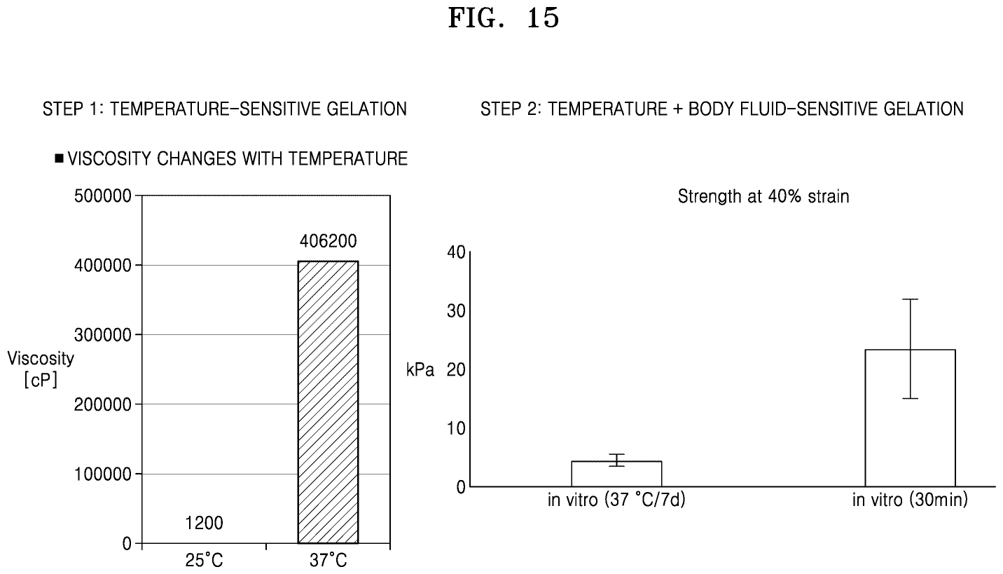

FIG. 15 is a diagram comparing the physical property of the hydrogel formed intradermally in the mouse after mixing the first agent and second agent of the liquid formulation (separate type) and the hydrogel formed in vitro after mixing the first agent and second agent of the liquid formulation and gelating in vivo, and the hydrogel formed intradermally in the mouse after fluid-sensitive gelation (integral type).

### Experimental Example 5. Evaluation of Persistence of Temperature-Sensitive Hydrogel

In the present experimental example, a persistence of temperature-sensitive hydrogel formed under in vivo conditions was evaluated. Specifically, the hydrogel composition of a liquid formulation, which was a mixture of the first agent and second agent, was stored in an incubator at 37 ºC for about 24 hours to proceed with gelation. Afterwards, the formed hydrogel was then cut into 0.2 ml portions, placed in 1.5 ml of PBS, sealed, and stored in a 37° C. incubator. Then, over time, the volume of each hydrogel was measured while removing the PBS.

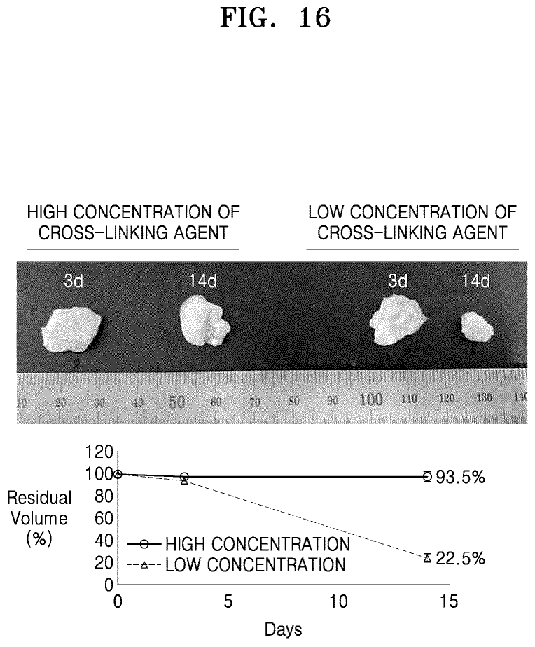

FIG. 16 shows a results of comparing a degradation rate according to a cross-linking agent concentration of the hydrogel formed intradermally in the mouse after mixing the first agent and second agent of the liquid formulation according to an example. As shown in FIG. 16, it was confirmed that the degradation rate of the hydrogel was slower when the concentration of the crosslinking agent was high.

### Experimental Example 6. Confirmation of Gelation of Hydrogel Including Decellularized Matrix

In the present experimental example, when a decellularized matrix was included in the hydrogel, an experiment was performed with the expectation that the ability to induce vascularized adipose tissue would be excellent. Specifically, the first agent included a decellularized matrix. Before injection into the mouse, the first agent and second agent were mixed and then injected into the body of the mouse to confirm gelation of the hydrogel under in vivo conditions.

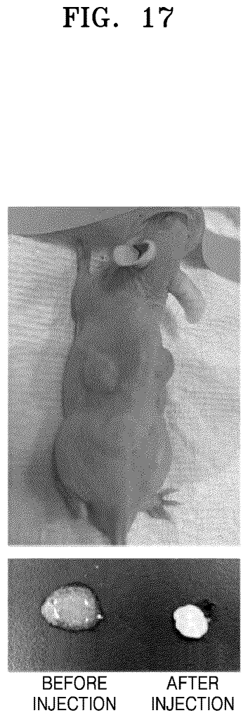

FIG. 17 is a diagram illustrating an observation of an appearance of the hydrogel before and after injection into a mouse after mixing the first agent and second agent of the liquid formulation including the decellularized material according to an example, and the mouse injected with the first agent and second agent of the mixed liquid formulation. As shown in FIG. 17, it was confirmed that gelation progressed excellently under in vivo conditions.

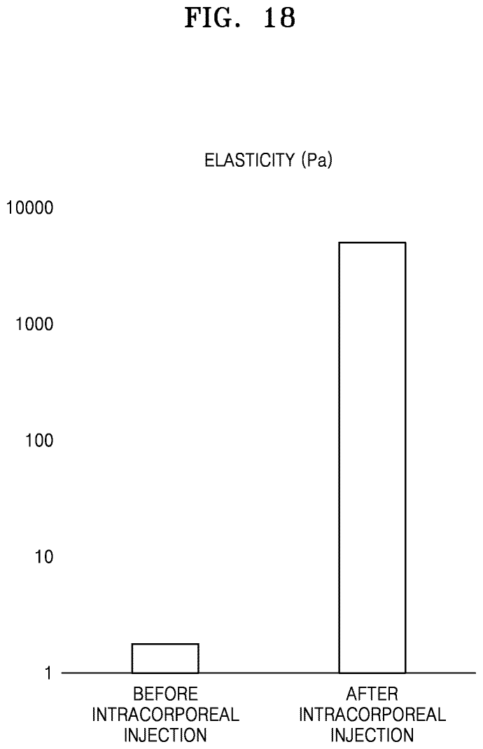

FIG. 18 shows a results of confirming the elasticity of the hydrogel before and after injection into the body as confirmed in FIG. 17 above. As shown in FIG. 18, it was confirmed that the elasticity increased significantly when injected into the body compared to when the first agent and second agent including the decellularized matrix were simply mixed. Therefore, it was confirmed that the hydrogel including the decellularized matrix achieved a remarkable level of gelation in response to in vivo conditions.

The foregoing description of the present disclosure is for illustrative purposes only, and one that has ordinary skill in the art to which the present disclosure belongs will understand that the present disclosure may be readily adapted to other specific forms without altering the technical ideas or essential features of the present disclosure. Therefore, the examples described above should be understood in all respects as illustrative and not restrictive.

1. A composition for preparing a temperature-sensitive hydrogel composition,
comprising a first agent in a liquid formulation comprising an aqueous solution containing chitosan and phosphate ions,
wherein the composition comprises the aqueous solution containing 0.05 wt % to 3.5 wt % of chitosan and 0.1 wt % to 40 wt % of phosphate ions based on the total weight of the first agent, and
the weight ratio of an aqueous solution containing chitosan to an aqueous solution containing phosphate ions comprised in the first agent is from 1:0.01 parts by weight to 1:0.3 parts by weight.

2. The composition of claim 1, wherein the first agent in the liquid formulation has been stabilized at room temperature conditions for 1 hour to 7 days.

3. The composition for preparing a temperature-sensitive hydrogel composition, of claim 1,
wherein the aqueous solution containing phosphate ions comprises at least one or more phosphate selected from the group consisting of sodium phosphate dibasic, sodium phosphate monobasic, ammonium phosphate dibasic, dihydrogen phosphate, trisodium phosphate, potassium phosphate dibasic, potassium phosphate monobasic, dimethyl phosphate, monomagnesium phosphate, magnesium phosphate dibasic, lithium dihydrogen phosphate, lithium phosphate, calcium dihydrogen phosphate hydrate, and calcium hydrogen phosphate.

4. The composition for preparing a temperature-sensitive hydrogel composition, of claim 1, wherein a weight ratio of the aqueous solution containing chitosan to the aqueous solution containing phosphate ions is from 1:0.16 to 1:0.18.

5. The composition of claim 1, wherein the first agent in the liquid formulation further comprises a decellularized matrix.

6. The composition for preparing a multi-crosslinked temperature-sensitive hydrogel composition, of claim 1, further comprising a second agent in a liquid formulation containing glycerol.

7. The composition of claim 6, wherein the second agent in the liquid formulation further comprises a decellularized matrix.

8. The composition for preparing a multi-crosslinked temperature-sensitive hydrogel composition, of claim 6, wherein the second agent comprises 0.10 parts by weight to 0.14 parts by weight of the glycerol per 1 part by weight of the aqueous solution containing chitosan of the first agent.

9. The composition for preparing a temperature-sensitive hydrogel composition, of claim 1,
wherein the composition further comprises a third agent comprising a basic aqueous solution for changing strength of the hydrogel composition.

10. The composition for preparing a temperature-sensitive hydrogel composition, of claim 6,
wherein the first agent and the second agent are isolated in separate spaces within a container, and
a mixture including the first agent and the second agent forms a gel formulation after being injected into a body.

11. The composition for preparing a temperature-sensitive hydrogel composition, of claim 6, wherein
the first agent and the second agent are in a mixed state, and
a mixture including the first agent and the second agent forms a gel formulation before being injected into a body.

12. A method of preparing a temperature-sensitive hydrogel composition, the method comprising: preparing a first agent in a liquid formulation by mixing a solution containing chitosan ions and an aqueous solution containing phosphate ions,
wherein a weight ratio of the aqueous solution containing chitosan ions and the aqueous solution containing phosphate ions in the first agent is from 1:0.01 parts by weight to 1:0.3 parts by weight.

13. The method of preparing a temperature-sensitive hydrogel composition, of claim 12, further comprising stabilizing the first agent in the liquid formulation for 1 hour to 7 days.

14. The method of preparing a temperature-sensitive hydrogel composition, of claim 12, further comprising mixing the first agent in the liquid formulation with a second agent in a liquid formulation containing glycerol.

15. The method of preparing a temperature-sensitive hydrogel composition, of claim 14, further comprising multi-crosslinking a liquid formulation mixture including the first agent and the second agent under in vivo conditions.

16. The method of preparing a temperature-sensitive hydrogel composition, of claim 12, further comprising mixing a third agent including a basic aqueous solution for changing strength of the hydrogel composition.
